(19) United States
(12) Patent Application Publication
(10) Pub. No.: US 2014/0058238 A1
(43) Pub. Date: Feb. 27, 2014

(54) COLOR-CODED ARRAY OF SUBDERMAL RIBBON NEEDLE ELECTRODES FOR SURGICAL MONITORING

(71) Applicant: ROCHESTER ELECTRO-MEDICAL, INC., (US)

(72) Inventors: Lisa B. Novorska, Lutz, FL (US); Mark Berkins, Land O' Lakes, FL (US)

(73) Assignee: ROCHESTER ELECTRO-MEDICAL, INC., Lutz, FL (US)

(21) Appl. No.: 13/761,945

(22) Filed: Feb. 7, 2013

Related U.S. Application Data
(60) Provisional application No. 61/595,878, filed on Feb. 7, 2012.

(51) Int. Cl. A61B 5/04

(52) U.S. Cl. CPC A61B 5/04001
USPC 600/373; 29/825

(57) ABSTRACT

The current invention discloses a ribbon strand of four (4) or more color-coded needle electrodes customized for a particular surgical procedure. Each needle electrode includes a needle, a wire with an electrical lead, and an electrical connector. The array of needle electrodes is abuttingly coupled in a linear formation to form a ridged planar surface. The needle and wire of each needle electrode have a predetermined set of dimensions (e.g., length) based on the surgical procedure. The wire of each needle electrode can also be colored to indicate position of application of that needle electrode.

### BACKGROUND OF THE INVENTION

1. Field of the Invention

This invention relates, generally, to needle electrodes. More particularly, it relates to an array of color-coded ribbon electrodes used for monitoring surgical procedures.

2. Description of the Prior Art

Needle, cup and surface electrodes are all well-known in the art. Needle electrodes have been used previously to monitor intraoperative electrocardiography, though currently the most prevalent use is in intramuscular electromyography. Needle electrodes are inserted subcutaneously into muscle tissue to monitor evoked potentials, or evoked responses during medical procedures, including neurosurgical and orthopedic procedures. Needle electrodes may also monitor neural transmission pathways to ensure proper signaling and may also monitor electroencephalographic brainwaves to ensure proper brain function.

Typically, needle electrodes are inserted into a patient as single, separate wires each attached on one end to an outer shaft beveled to a sharp point and on the other end to a plug, or electrical connector, secured to an apparatus that depicts the electrical activity flowing through the needle. Thus, an issue arises when several needles are needed for placement into a patient, and each needle must separately travel to the monitoring apparatus, and each needle must be removed post operation while holding individual wires. The wires hinder the physician and technicians' ability to complete the medical procedure safely, effectively and efficiently. Significantly, use of single wires causes artificial waves to be seen on the monitoring apparatus, as there is a greater degree of movement by the wires individually (i.e., electrode sways and popping). Use of single wires creates a higher risk of monitoring errors caused by crossing of wires and electrodes. Separate packaging for wires and electrodes also becomes more expensive to manufacture,

If needles are required to be placed in close proximity to each other on the patient's body, the current state of the art would force a technologist to use multiple separate wires very close to one another in order to use several different needle lengths in the same localized area of the body. This positioning increases artifact and the risk of pulling out one of the needles. Positioning of individual wires and needles also increases the risk of the wires wrapping around the extremities of a patient or getting caught on structures in the external environment.

Moreover, medical tape can be used to secure wires to a patient's body or other apparatus, but this becomes time-consuming and tedious for the surgeon and technicians in prepping a patient for surgery when every minute of time is crucial to stay on schedule, and every minute of surgery time is very expensive.

Additionally, if the wires have only a couple varied colors, it becomes more time-consuming to sort the wires and keep track of the wires. This increases the skill needed to assemble the electrodes and prep the patient, which, in turn, increases healthcare costs. This also increases the risk of monitoring error if electrodes and wires are mistaken, considering that some electrodes, according to the specific protocols required by a practitioner, facility, or procedure, might be utilized for certain parts of the body, while other electrodes might be utilized for other parts of the body. If a surgical procedure requires “passing off” of monitoring duties to another technologist (e.g., taking a break, switching duty shifts, etc.), then the subsequent technologist must spend extra time sorting through each wire, color and body region to determine the state of electrical connection between the patient and the monitoring apparatus.

Further, single or pairs of needle electrodes run the risk of being pulled out of the patient more easily as individuals must maneuver around several wires of the singular or paired electrodes. Thus, when needles are pulled out erroneously, the contaminated needles may stick other individuals or may become further contaminated by touching objects or surfaces in the external environment. Singular or pairs of needle electrodes must often be opened and knotted together prior to surgery, further increasing risk of contamination.

Accordingly, what is needed is a color-coded, ribbon of electrodes for intraoperative monitoring. However, in view of the art considered as a whole at the time the present invention was made, it was not obvious to those of ordinary skill how the art could be advanced.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

The long-standing but heretofore unfulfilled need for an improved, more effective and lower cost apparatus of needle electrodes is now met by a new, useful and nonobvious invention.

An embodiment of the current invention includes a ribbon strand of four (4) or more electrodes, comprising an array of color-coded needle electrodes. Each needle electrode includes a needle, a wire with an electrical lead, and an electrical connector. The needle, wire portion with electrical lead, and electrical connector maintain an electrical connection. The array of needle electrodes is abuttingly coupled in a linear formation to form a ridged planar surface. A plurality of optional adhesives, labels, and/or printed measurements may be disposed on at least one side of the ridged planar surface to facilitate attachment of the ribbon strand to a patient's body or other objects in the surgical room.

The color-coded needle electrodes can be personalized to a user or to a surgical procedure. The personalization concerns number of needles, length of needles, length of electrical leads, colors and arrangement of optional adhesives, labels and/or printed measurements.

The ribbon strand may include a distal enclosure connecting the needle and the electrical lead to maintain a secure connection for displaying electrical activity stemming from the needle. The distal enclosure may have a color that is the same as the wire with which it is contiguous.

The electrical connector may have a color that is the same as the wire with which it is contiguous.

The ribbon strand may include a needle protector disposed in outer relation to the needle. The needle protector would have a length longer than the length of the needle.

The electrical connector may have a ridged surface to facilitate user manipulation of the electrical connector.

The wire of each needle electrode may be conformed in a ribbon along a majority of its length and then separate at its proximal end and at its distal end.

The plurality of adhesives may be disposed on both sides of the ridged planar surface of the ribbon strand to facilitate attachment of the ribbon strand to a patient's body or to other objects in the surgical room.

In a separate embodiment, the current invention includes a method of monitoring a surgical procedure. The steps of this method comprise first assembling a ribbon strand including an array of color-coded needle electrodes. The array is arranged in a linear formation to form a ridged planar surface. The ribbon strand may further include a plurality of adhesives, labels, and/or printed measurements disposed on at least one side of the ridged planar surface. Next, each needle electrode of the array of needle electrodes is inserted into a patient's body. Each electrical connector of the array of needle electrodes is inserted into corresponding outlets of a neurological monitoring apparatus. The neurological monitoring equipment can be activated, and evoked potentials can be displayed on and read off of the neurological monitoring equipment. Based on these readings, the surgical procedure can be monitored and adjusted.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the nature and objects of the invention, reference should be made to the following detailed disclosure, taken in connection with the accompanying drawings, in which:

FIG. 1A depicts a needle electrode in use as seen in the prior art.

FIG. 1B depicts a plurality of needle electrodes in use as seen in the prior art.

FIG. 1C depicts a plurality of needle electrodes in use during an EEG procedure as seen in the prior art.

FIG. 2A is an upper perspective view of a distal end of an embodiment of the current invention.

FIG. 2B is a frontal view of the embodiment of FIG. 2A.

FIG. 3 is a top view of a ribbon strand with optional adhesives attached to a ridged planar surface formed by the linear formation of the coupled leads.

FIG. 4 is a top view of a middle portion and distal end of an embodiment of the current invention. The view depicts separation of the leads at the distal end and optional adhesives attached to a ridged planar surface of the leads when coupled to each other along the ribbon strand's middle portion.

FIG. 5 is a top view of a distal end of an embodiment of the current invention.

FIG. 6 is a top view of a coupled wire portion and a distal end of an embodiment of the current invention.

FIG. 7 is a top view of a proximal end of an embodiment of the current invention.

FIG. 8A is a perspective view of a ribbon strand of leads depicting placement of a label.

FIG. 8B is a perspective view of the ribbon strand of FIG. 9A with label attached to a ridged planar surface of the ribbon strand.

FIG. 9 is a perspective view of a ribbon strand of leads with ruler printed on a ridged planar surface of the ribbon strand.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

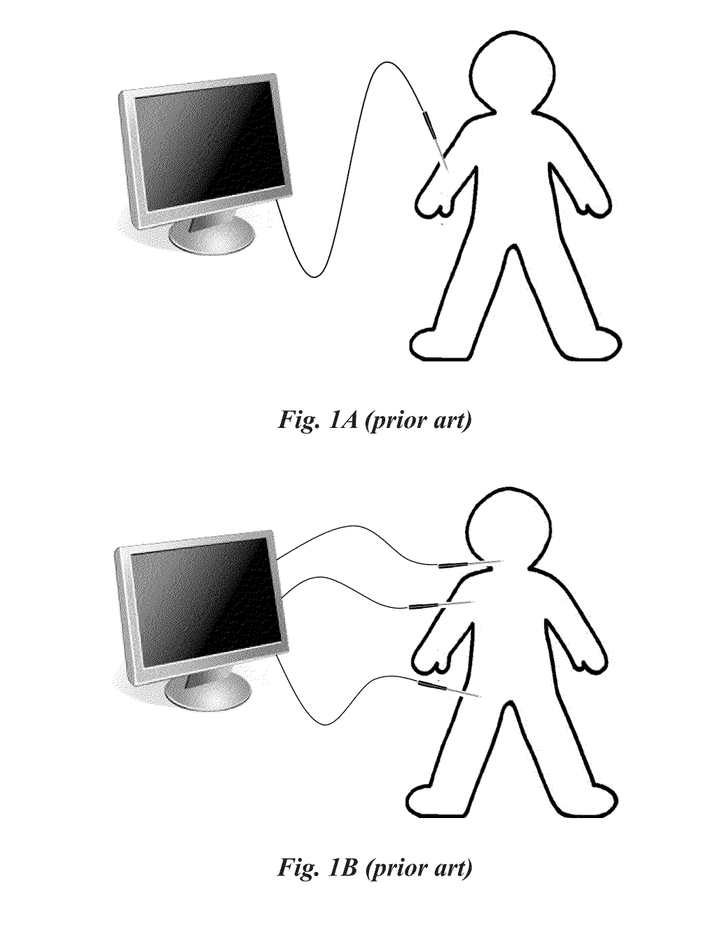

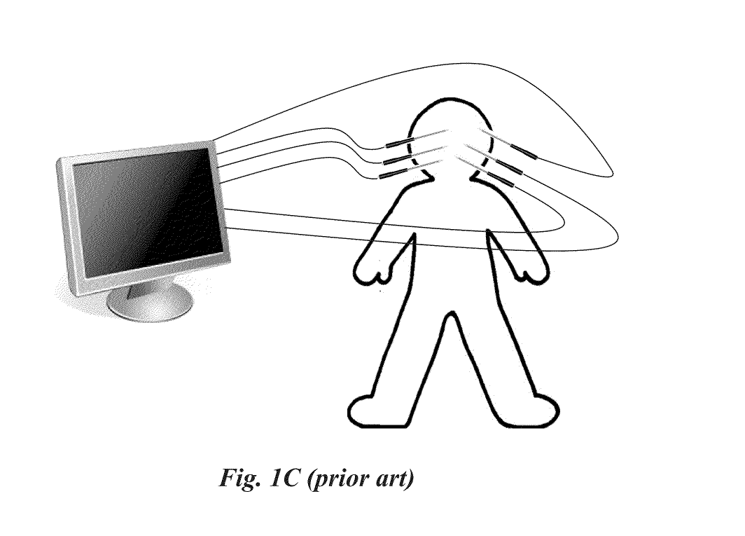

FIG. 1A depicts a conventional needle electrode apparatus containing one needle, one wire and one plug. Movement of this needle electrode apparatus while in use, as depicted in FIG. 1A, produces artificial sways and popping, which, in turn, causes the apparatus displaying the electrical activity, to display artificial waves that are not actually occurring within the patient's body. FIG. 1B depicts the conventional method of using multiple needle electrodes to measure electrical activity within a patient while performing electromyography. The three needle electrodes remain separated as the wire portions travel from the needle to the plug into the monitor. Slight movement in any of the wire portions or needle would cause artificial sways and popping to appear on the monitor, thus skewing the data received from the electromyography. Moreover, the wire portions traveling separately to the monitor may interfere with the movement of the persons in the operating room, increasing risk of accidental contact with the wire portions or needle. FIG. 1C depicts six (6) single wires attached to the head of a patient monitoring electroencephalography, as seen in the prior art. This type of monitoring is continuous through certain surgeries and is a very typical place/procedure that induces swaying and popping since the signal is recorded and analyzed continuously during the procedure.

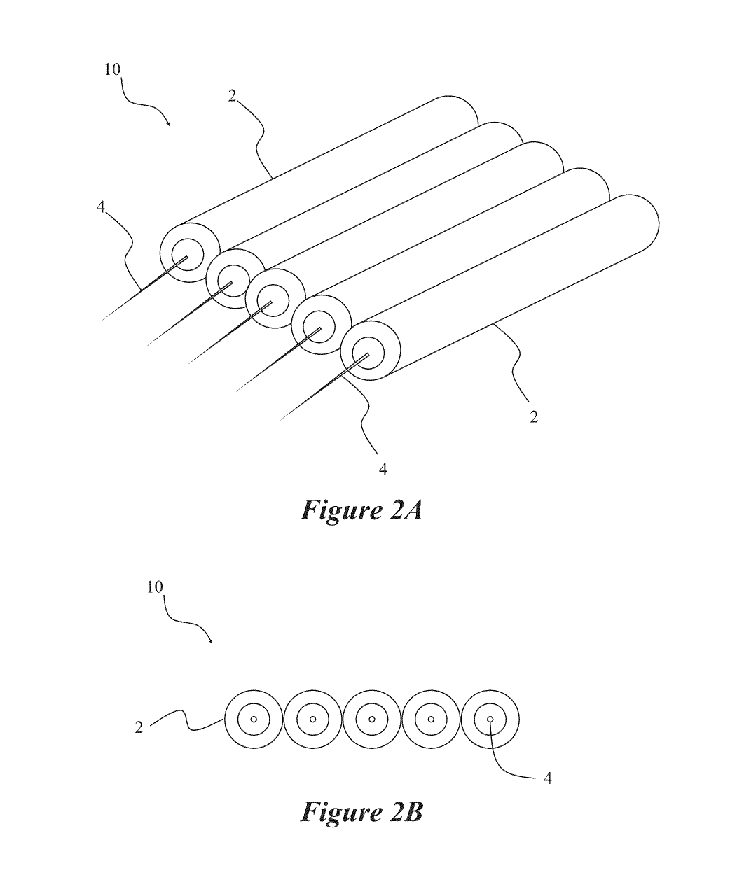

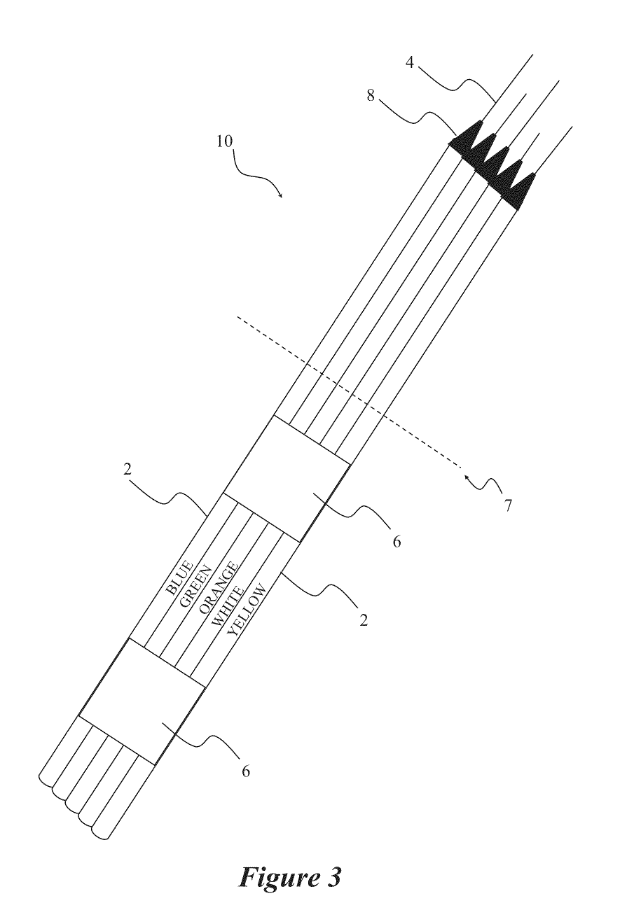

As depicted in FIGS. 2A and 2B, certain embodiments of the current invention include a ribbon strand of an array of needle electrodes, denoted generally by the reference numeral 10, inserted subcutaneously during surgery to monitor evoked potentials or encephalography during the surgical procedure. The muscular or neural responses to the surgical procedure are read by needle electrodes 4 and transmitted via electrical leads within wires 2 to the apparatus (not shown) that displays the muscular activity, or neurological monitoring equipment, for example a computer and monitor. An array of needle electrodes 10 needed for a surgical procedure is abuttingly coupled to each other at a majority portion of wire portion 2 of each needle electrode. Wires 2 are attached in a substantially linear formation to create a ridged planar surface, as depicted in FIG. 2B. Attaching an array of wires 2 in this manner reduces the errant artificial waves (i.e., sways and popping) that are produced by using single, separate wires. These errant waves appear even if just a couple of wires are attached to each other. For example, at least four or more wires might be needed to maintain consistency in the waves. However, less than four wires might be necessary in a given specific locality to alleviate issues of wires bumping into each other. As used herein, the term “array” refers to a number of wires or electrical leads used within a needle electrode apparatus sufficient both to satisfy the need of a localized monitoring site without requiring more non-arrayed needles, and to eliminate sway and popping in order to maintain consistency of the waves.

As noted, the wires of the needle electrodes are attached along a majority of their lengths to create a ribbon with a ridged planar surface along the attached portion. The wires have a cylindrical shape, seen in FIGS. 2A and 2B, and are flexible. Other shapes are contemplated, though, for ease of manufacture and/or use, if desired or needed by the user. As used herein, the term “distal end” refers to an end of the needle electrode or wire portion that includes the needle. As used herein, the term “proximal end” refers to an end of the needle electrode or wire portion that includes the plug or electrical connector that is secured to the neurological monitoring apparatus. The proximal and distal ends of the wires each have a segment wherein the wires are not abuttingly coupled to each other. Rather, the wires remain singular and separated, so the needles and the plugs can be manipulated without restraint of attached wires. Dashed line 7 in FIG. 3 depicts an example of location, proximal to which the wires are coupled and distal to which the wires are separated. Needles may be easily placed in various localized regions of a patient's body. The same concept applies to the plugs on proximal end of the needle electrode. Plugs may be easily placed into the neurological monitoring apparatus.

As depicted in FIG. 3, an optional plurality of adhesives 6 can be disposed on the ridged planar surface of the ribbon to create a flat planar surface, as depicted in FIG. 2B. Adhesives 6 may be disposed at set intervals along ribbon 10, or may be disposed in custom configurations as needed or desired by a user. Adhesives 6 may be disposed on only one side of the ridged planar surface of ribbon 20 or on both sides of the ridged planar surface of ribbon 20. A peel away strip (not shown) is disposed in overlying relation to each adhesive 6, so when a user removes the peel away strip (not shown), the adhesive properties of corresponding adhesive 6 are exposed.

Once adhesive properties are exposed, optional adhesives 6 are capable of securing ribbon 10 to a patient's body and other apparatus in the surgical room, such as the bed frame or other available objects. Thus, adhesive 6 should be of a type that is safe and secure for attachment to both a patient's skin and inanimate apparatus. Securing ribbon 10 to a patient's skin or to surrounding apparatus alleviates the hindrance of ribbon 10 for persons moving around the surgical room. Moreover, if contact is accidentally made with ribbon 10, adhesives 6 alleviate the effect on needles 4, further reducing any artificial waves that may be produced. Contact with wires 2 would pull on the nearest distal adhesive 6 rather than pull on needles 4, allowing needles 4 to remain secure within the patient's body.

Additionally, wires 2 coupled in ribbon formation are color-coded, as depicted in FIG. 3 by the word indicating color of each wire, each color specific to a particular part of the body in a particular surgical procedure. Thus, rather than having to determine whether a needle electrode belongs in a certain body region or having to match particular needle electrodes, each color indicates where each electrode belongs. Plugs on the proximal end of the needle electrode may also have the same color as each plug's corresponding wire. Color-coded needle electrodes reduce human error, skill needed to assemble the apparatus and prep the patient, healthcare costs, time needed to prep the patient, and risk of contamination. An embodiment of the invention may include a legend, or key (not shown), that correlates each color to each region of the body where the corresponding electrode should be utilized. Color-coding helps significantly reduce monitoring error and helps provide error-free monitoring continuity when a technologist must “pass off” the monitoring duties to another technologist (e.g., taking a break, switching duty shifts, etc.), among other benefits. This decreases the skill needed to assemble the electrodes and prep the patient, which, in turn, decreases healthcare costs.

Needles 4 included within needle electrodes 10 may be of any size required by the user, as depicted in FIG. 3. Each needle 4 within ribbon strand 10 may be the same size. Alternatively, each needle 4 within ribbon strand 10 may be different sizes, depending on the desires of the user and/or requirements of the procedure for which ribbon strand 10 was designed. Needles 4 are beveled to form a sharp distal-most point.

The density and depth of muscle tissue, along with other factors, might require the use of longer or shorter needles 4 in different locations around the patient's body, or even in varying specific insertion sites that are in the same general location and are within close proximity to each other. The ability to mix and match the lengths of needles 4 within array 10 is specifically advantageous, particularly when needles 4 must be placed in close proximity to each other on the patient's body. Maintaining needles 4 in array 10 decreases artifact and the risk of pulling out one of the needles.

Distal enclosure 8 encloses distal end of wire 2 and proximal end of needle 4, such that electrical leads within wire 2 remain securely in contact with needle 4 to accurately transmit electrical activity received by needle 4. Distal enclosure 8 has an inner diameter larger than an outer diameter of wire 2, such that wire 2 fits snugly within the inner diameter of distal enclosure 8. Distal enclosure 8 has an inner diameter larger than an outer diameter of needle 4, such that needle 4 fits snugly within the inner diameter of distal enclosure 8. Needle 4 may be soldered or crimped to wire 2 to ensure this connection. Distal enclosure 8 is designed for aesthetics and additional connection security, but typically is not primarily used to attach or reinforce wire 2 to needle 4. The soldering and/or crimping primarily secures needle 4 to wire 2. Other methods of connection between needle 4 and electrical leads within wire 2 are contemplated as well.

Certain embodiments of the current invention can be personalized to each user. For example, if a surgeon requires electrode kit 10 for a spinal fusion procedure for a particular patient, the current invention can be produced with the appropriate number of electrodes in each ribbon 10, each electrode being color-coded for the region of the patient's body where the electrode belongs and each electrode having the appropriate length of needle. Alternatively, the current invention can be sold to users as a predesigned kit sold for particular uses. Thus, there may be specific kits for spinal fusion procedures that various surgeons could use on any patient in need thereof. Each of these kits or apparatuses would be packaged together, thus significantly increasing time-efficiency and reducing waste by using a single, sterilized package for a procedure.

Ribbon strand 10 may also be disposable, such that after each surgical procedure, ribbon strand 10 can be entirely disposed. Standard needle electrodes may be used within ribbon strand assembly 10.

Alternatively, ribbon strand 10 can be sold as a larger kit containing an array of needle electrodes, each needle electrode coupled together along the wire portion. For example, ribbon strand 10 can include sixteen individual needle electrodes coupled together in linear formation to form a ridged planar surface. Groupings of needle electrodes can be peeled off of the larger array for use during a surgical procedure. For example, if a kit contained sixteen needle electrodes and six needle electrodes were needed for a spinal fusion procedure, the appropriate six needle electrodes could be peeled off the larger array as a group or individually.

Unexpected Benefits

A ribbon strand containing at least four (4) needle electrodes provides a plurality of benefits that were not foreseen at the time of invention. In a preferred embodiment, the ribbon strand has more than four (4) or five (5) needle electrodes and less than about ten (10) needle electrodes. If more than ten (10) needle electrodes are used in one (1) ribbon strand, the ribbon strand may become unnecessary during a surgical operation because of its size, weight (when it is suspended in the air leading from the computing system to the patient), and number of electrodes interfering with the procedure. However, there absolutely are certain uses for more than ten (10) electrodes in a single ribbon strand if desired by a particular healthcare facility. The range of four (4) or five (5) to ten (10) electrodes in a ribbon strand is intended to be exemplary, rather than limiting. The current invention does contemplate more than ten (10) electrodes in a single ribbon.

The ribbon strand consolidates all needle electrodes needed for a single surgical procedure along a patient's extremities or other specific location (e.g., the patient's head). Thus, each ribbon strand is purchased and immediately ready to use for a surgical procedure. Several ribbon strands may be used for a single surgical procedure, while still providing the same benefits described herein. For example, there may be a specific ribbon for the head, a ribbon for the left leg, a ribbon for the right arm, etc. A typical surgical procedure, such as a spinal fusion for example, may require 40+ needle electrodes, thus possibly requiring five (5) ribbons containing eight (8) leads per ribbon. Other procedures, such as a carotid endarterectomy, only require monitoring of the patient's electroencephalography signals on the head, thus possibly requiring only one (1) ribbon of leads.

The ribbon strand is customizable to a user or technician's needs for a surgical procedure. Each ribbon might have between four (4) and ten (10) leads (i.e., needle electrodes) depending on the area of the patient's body for which the ribbon is being used and/or on what tests are intended to be implemented during each specific surgery/procedure. Further, the color(s) of each lead can be customized within each ribbon based on use of that lead in that particular procedure. Particular colors may be placed adjacent to each other as well to indicate particular uses of the adjacent leads, so order of the colors can become important. The colors can indicate active and/or reference recording points. This allows technologists to color code ribbons based on muscle groups and cascade the colors by relation of the muscle to one another. Colors do not need to be paired with black and white, as the conventional art teaches is a requirement. The paired subdermal needle electrodes, seen in the prior art, pair a colored lead with a black or white lead because they were designed for use on the extremities where an active lead and a reference lead were needed for each muscle group. Black and white were chosen primarily because of a lack of color options and because they contrast so extremely, so it would be more difficult to confuse a color-white electrode pair on the right side of a patient with a color-black electrode pair on the left side of the patient. For instance, a specific muscle group in a patient's right leg could be assigned “red” as its active color and white as its reference, and the corresponding muscle group in the patient's left leg could be assigned “red” as its active color and black as its reference. It is generally taught that “white is right” and “black is left.” These types of pairs are not currently advantageous for use in other locations, such as the head, however. For the head, having several white or black electrodes (as parts of pairs) would be very confusing and dangerous. Using conventional electrodes, most people would not be able to use paired electrodes for the head at all, and would need to resort to using individual electrodes, which would present the possibility of using a plurality of different colors needed to safely monitor this area. However, using individual electrodes would not provide any of the advantages of a non-single configuration, such as a ribbon strand of the current invention. The current ribbon configuration would facilitate a technician's ability to trace which white or black reference corresponds with each muscle group, and also allows the technician to see each group in cascading order “up” or “down” the extremity. Additionally, in certain facilities, intraoperative monitoring departments have previously adopted the practice of assigning the same color for the active and reference electrodes of muscle groups. In this case, two red electrodes would be placed in one muscle group and just tracked as to which is the active and which is the reference. In some cases, those facilities have not been able to switch to paired electrodes, because their color schemes were not offered, since the prior art dictated only the general use of pairing with black and white. The current ribbon strand would alleviate this issue by having a personalized, color-coded ribbon strand of electrodes.

Additionally, ribbons can include leads in different lengths (i.e., for different parts of the body), whereas the conventional art teaches identical lengths for pairs of leads. For example, it would be advantageous to have leads of different lengths in one ribbon if one lead needs to be applied to the upper part of the leg and another lead needs to be applied to the foot. Placement of machine input boxes varies in different facilities. It is not uncommon for a box to be positioned at one end of the patient's bed. If, for example, the box is placed at the head, the distance needed to reach the patient's ankle likely is significantly different than the distance needed to reach the patient's waist. Different lengths would eliminate unwanted and wasteful slack, reduce the risk of artifact, and decrease set up time. Additionally, entire ribbons can be made of different lengths from other ribbons to obtain at least a portion of the same advantages.

Another customizable aspect of certain embodiments of the current invention is that needle lengths of leads within the same ribbon can have differing lengths. In order to obtain the best recordings possible, a needle must be able to traverse the adipose tissue and into the muscle. Thus, in some patients (especially the thigh portion), a standard 12 mm needle would not be adequate length to reach the muscle. Depending on use of each lead within the ribbon, needle length of each individual lead can be the standard 12 mm, longer than 12 mm, or shorter than 12 mm. Additionally, entire ribbons can have needles of the same length but different from other ribbons to obtain at least a portion of the same advantages.

A technician can also customize the pull on each lead within a single ribbon. Though all wires are linearly abuttingly coupled to one another, each lead can be singularly manipulated by peeling it away from the ribbon without compromising the wire or conductivity. For example, if a technician is using an 8-lead head ribbon, the six innermost wires can be slightly peeled apart in order to be placed in specific derivations on the patient's head, but the two outer wires can be peeled back much further for extra slack since they are inserted near the neck. Thus, the technician can easily conduct the procedure without the disadvantages seen in the prior art (i.e., slack in the wires, etc.), described previously.

An important advantage imparted by the current invention is that it maintains sterility through the surgical procedure. In many instances using conventional needle electrodes assemblies, technologists had opened and knotted pairs of electrodes together prior to surgery, failing to maintain sterility of the needles. This typically occurs because technologists are not given enough time in the operating room to prepare the electrodes on the patient. Contrastingly, technologists could use embodiments of the current invention immediately in the sterile field without opening and knotting electrodes together, thereby maintaining sterility of the needles.

Use of the current invention saves time before, during, and after surgery. Before surgery, less time is needed to open the wires, organize the wires, and find the wires to plug or insert into the body. During surgery, errors or artifact can be more quickly identified and prevented since wires are less likely to get caught on any external structure or wrap around the patient, causing the wires to be pulled, fall out, or have artifact. If any problem is found, the wires can be more easily found and the problem alleviated. After surgery, wires are easier to find and handle since they are coupled distally to form the ribbon aspect. Because of the customizability of the entire ribbon (e.g., length, number, etc.), the needle ends have less slack, facilitating manageability of the needles and reducing the possibility of wires swinging or pulling back leading to needles injuring someone erroneously or spreading contagions or bodily fluids from the patient to the healthcare workers present in the operating room.

The ability for a single ribbon of needle electrodes to be used for one procedure or a portion of one procedure minimizes the possibility of tangles, which provides a plurality of significant benefits. Wires and electrodes free of tangles decreases pre-surgery preparation time, meaning there is no more wasted time spent knotting and twisting wires, which when done improperly, can compromise the wire without the technologist's knowledge. Further, pre-surgical insertion time is reduced, as application of the needles of the ribbon is simple and concise. Use of the ribbon, minimizing or eliminating tangles, significantly decreases the possibility of artifact due to swaying, tangles, and singular or paired wires hitting one another or getting caught on an external structure in the operating MOM.

An underappreciated issue that arises in the conventional art occurs when IOM technicians must be substituted mid-procedure (often in 10+ hour procedures). Confusion arises with regards to the connection between the electrodes and the patient. Using the current invention, the duties of the new technician are facilitated by reducing the confusion, since the new technician would be able to more easily determine which color of wire is associated with which extremity/muscle of the patient. This increases monitoring accuracy, decreases troubleshooting issues, providing continuity throughout the IOM team, increases ease of training new technicians, and provides more confident patient care.

Minimization or elimination of tangling further decreases the chances of a needle being pulled out of the patient or a plug getting pulled out of the jack box, thereby also decreasing the possibility of the technician being stuck by an errant needle. If a needle were pulled out of the patient's body, it would be attached to the ribbon and therefore easy to locate, not free-floating, and not contaminated by external environment (e.g., the ground).

Further, untangled leads decrease the removal time after the surgical procedure is complete. Each needle can be slowly removed from the skin of the patient along the planar surface of the skin while the technician is stabilizing (e.g., holding) the wire portion of the ribbon. Once the needles have been removed from the patient, the distal end of the ribbon (i.e., the end with the needles) can be excised directly into a disposal. This makes disposal quick and easy, unlike the disposal of single and paired subdermals as seen in the prior art. With single and paired subdermals, technicians often must untangle all of the single or paired wires from the plug end while the needles are still in the patient's body. Then technicians must take each needle out individually; otherwise, the tangled wires wrap around each other and increase changes of technicians being errantly stuck by the free end of the needles.

Coupled together linearly, the four (4) or more leads create a ridged planar surface that has many significant benefits. The planar surface decreases friction by sliding across surfaces instead of becoming entangled or caught on external surfaces within the operating room. The planar surface also facilitates location of all wires of the ribbon. Paired or single wires, as seen in the prior art, are thin and flimsy, making them difficult to see in the operating room bed, which is often disarranged with sheets, pressure cuffs, catheter tubing, intravenous tubing, and many other apparatuses attached to the patient and/or bed. During surgery, a technician must be able to locate all electrodes and associated wires (during insertion, during the flipping of the patient's body (for spinal surgery, for example), during a malfunction (whether from artifact or problem during surgery), and at the end of surgery), and the prior art does not allow a technician to achieve this easily.

Further, the planar surface facilitates handling and control of the ribbon. This is important to enhance patient care, as the ribbon can be taped down or tucked under the patient more effectively and comfortably. Tape can also be added underneath the ribbon. Thickness of the ribbon allows a technician to adhere looped flat tape or double-sided tape to the ribbon. Ribbons are able to lay flat when pulled apart. The thickness and flatness of the ribbon also reduce risk of the wires wrapping around patient extremities. It is less likely for a flat, heavy, wide, and stiff surface to wrap around a toe, leg, finger or arm than a flimsy, easily bendable single or paired subdermal. This can happen in the operating room without the technician's knowledge and can cause a lack of circulation in the area, bruising, and/or a friction rash. This is more likely to happen during longer surgeries (e.g., spinal and brain surgery) monitored by IOM technicians.

The planar surface of the ribbon also reduces number of artifacts and facilitates identification of artifacts if they occur. Many operating rooms are electrically hostile environments. Often, a single lead or a pair of leads can cross another wire and pick up an artifact in a muscle channel on the technician's computer. A technician must then identify whether the signal is an artifact or an actual biological change. Contrastingly, if the current invention (a ribbon) crosses over a wire that causes artifact, all leads would be affected, and all channels would show the change, allowing that artifact to be easily identified as an extraneous artifact and not a biological change. The reason this occurs is because stress or tugging on the wires would be evenly distributed throughout all leads, also decreasing the possibility of plugs becoming disconnected from the jack box or needles sliding out of the body. Reducing the possibility of needles sliding out of the patient's body also decreases the amount of times the patient must be pricked with needles since needles are more likely to remain inside the patient's body. For example, when monitoring signals from the patient's head, needles are often stapled into the patient's head for security, so a needle falling out of the head would cause three more wounds that the patient would not have experienced if the needle had remained in the patient's head.

Needles sliding out of the patient's body often also cause technicians, nurses, anesthesiologists, doctors, and other healthcare workers to be pricked by the needles. Utilizing an embodiment of the current invention, though, it is less likely that a needle is accidentally pulled from the patient's body, a needle pulled from the body is still attached to the ribbon and not free floating in the surrounding environment (e.g., the patient's bed). This reduces the risk of contaminated needles sticking persons involved in the surgical procedure. For example, if two 2.5-meter single subdermal needles were pulled from a patient's body, they could be anywhere within 2.5 meters of the jack box. The technician most likely would need to find each needle from the plug end and trace up the wire. As such, the needle could easily stick an individual as the technician pulls the wire. This problem would only exist if all needles of a ribbon were pulled from the patient's body, meaning that if a ribbon includes five leads, all five leads would need to be pulled versus just one needle pulled as in the prior art. Even if all five leads were pulled, the needles would be free-floating, but only a small portion of the wires would be free-floating (i.e., the distal portion of the wires that were pulled apart, typically just a couple of inches). The structure, stiffness and form of the ribbon prevent a dislodged needle from swinging to the ground and becoming contaminated, thereby reducing the possibility of infection for the patient. This also decreases cost for the monitoring company and hospital.

Additionally, the ribbon of the current invention allows individual wires to have a higher weight since each wire is anchored by the other wires. The weight of the ribbon decreases the possibility of the wires rippling up and the possibility of slack falling off the bed or moving around, causing artifact and increasing risk of being pulled. The form and weight of the ribbon also stabilizes and stiffens the individual wires, thereby decreasing the amount of artifact seen.

The ribbon also would allow members of the surgery team to more efficiently work on the patient post-induction. Because there are many wires, tubes, etc. associated with a patient coming out of surgery, keeping leads together in a ribbon facilitates the post-induction process for the surgery team by increasing organization. Further, when the technologist needs to reposition the patient or otherwise move the patient, leads coupled together in a ribbon can easily be moved without becoming tangled or becoming a hindrance.

Certain embodiments of the current invention also have several manufacturing and packing benefits by being coupled in arrays of leads. A customized ribbon of leads for a particular surgical procedure is contained in one package. For example, a ten-lead ribbon would be in one package rather than ten (10) single packages or five (5) paired packages. It is not uncommon for needles for a single surgery to be in forty (40) individual packages. Because of the value of time in an operating room, opening one package per extremity, rather than 5-10 packages per extremity, is far more efficient. It is also less wasteful, benefiting hospitals attempting to reduce waste. This benefit could not be accomplished with single or paired subdermal needles without creating other negative implications. Even if a manufacturer chose to package many single or paired subdermal needles together in one package, removing the electrodes from the package would be a very difficult process, as the electrodes would become tangled, resulting in a significant loss of time and organization.

Further, ribbons of leads reduce cost of manufacturing, as packaging, labor, and labeling add excess costs onto the price of each needle or pair of needles. Using only one bag, one label, and one period of time to place the entire ribbon into a bag is more efficient and allows a supplier to decrease the cost of the ribbon. This decreased cost of manufacture and packaging allows a healthcare facility to customize the ribbons needed, which otherwise might be too expensive.

Additionally, empty packages are often left in the patient's bed during surgery because of the limitation on time during preparation. The exterior of this packaging and associated labels are not made for use in the operating room and are thus not sterile and not specifically hypoallergenic, etc. Having a low number of packages would facilitate the technologist's ability to keep track of them. Decreasing the chance of packages being left in the patient's bed during surgery helps maintain patient safety and success within the operating room.

### Example 1

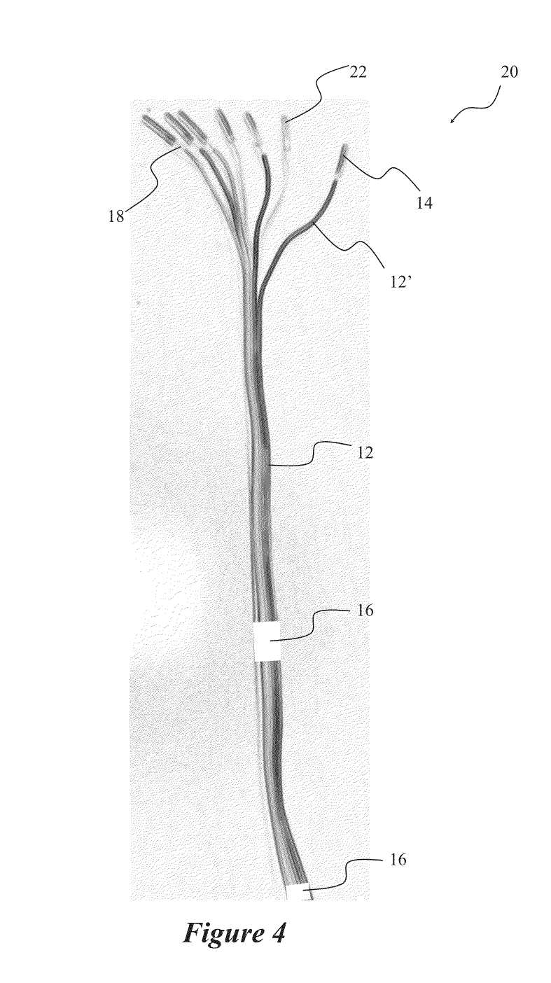

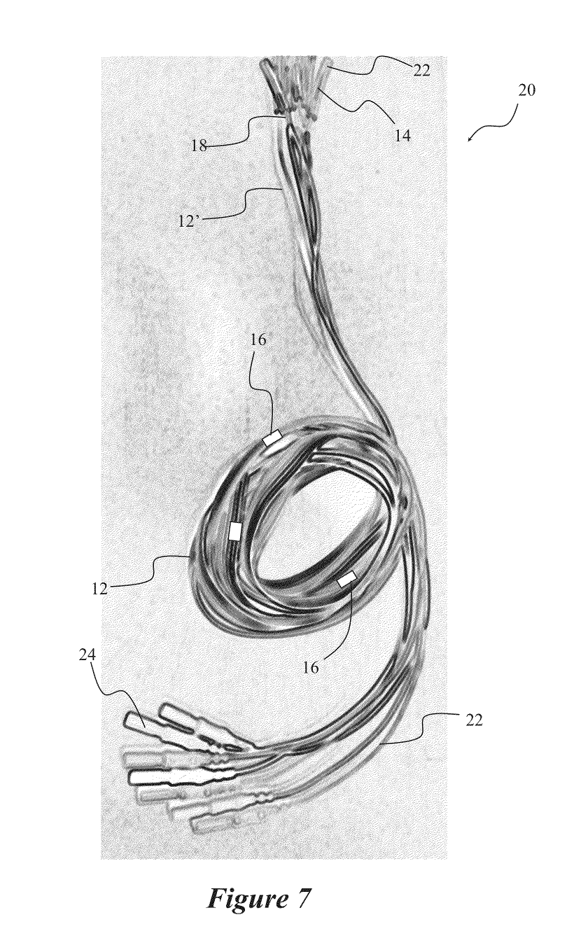

FIGS. 4-7 depict an embodiment of the current invention. An array of needle electrodes is arranged in configuration of ribbon 10. The distal end of each needle electrode includes needle 14. The proximal end of needle 14 is snugly enclosed within the distal end of distal enclosure 18. Thus, distal enclosure 18 includes a distal inner diameter larger than the proximal outer diameter of needle 14.

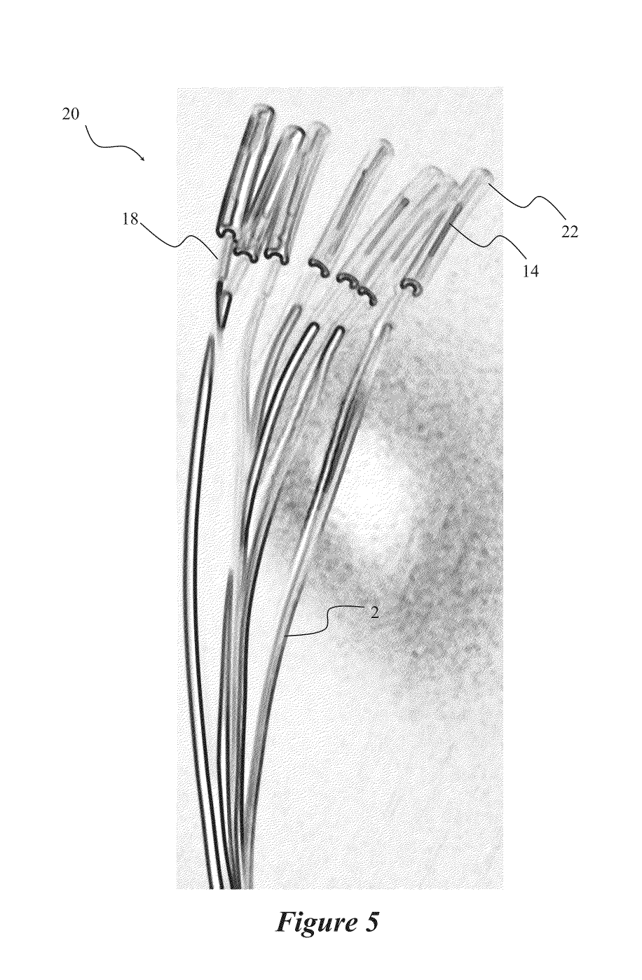

The embodiment may include needle casing 22, as depicted in FIG. 5, disposed in outer relation to needle 14 and distal enclosure 18, or a portion thereof. Needle casing 22 includes a proximal inner diameter larger than the distal outer diameter of distal enclosure 18, such that needle 14 fits snugly around the outer diameter of distal enclosure 18. Needle casing 22 has a length at least longer than the exposed portion of needle 14; thus, needle casing 22 ensures protection from the distal-most point of needle 14.

The distal end of each needle electrode is separate and singular, thereby facilitating placement of needles 14 to various regions of a patient's body. Wire portions 12 may then be coupled to each other, as depicted in FIG. 4, to form ribbon strand 20. Wire portions 12 are removably coupled in a linear formation to form a ridged planar surface, similar to that seen in FIG. 2B. Each wire portion 12 may be separated from the adjacent wire portion simply by applying a force of pulling them apart. Thus, for example, wire portion 12′ can have a longer separate segment, such that its corresponding needle electrode can reach a body region that is a further distance away from the remaining needle electrodes.

Wire portion 12 of each needle electrode is color-coded, specific to the needs of the user or specific to the procedure for which ribbon strand 20 was developed. Distal enclosures 18 may each be the same color or match the color of corresponding wire portion 12. Each color is specific to a particular part of the body in a particular surgical procedure. Thus, rather than having to determine whether a needle electrode belongs in a certain body region or having to match particular needle electrodes, each color indicates where each electrode belongs. Color-coded needle electrodes reduce human error, skill needed to assemble the apparatus and prep the patient, healthcare costs, time needed to prep the patient, and risk of contamination. An embodiment of the invention may include a legend, or key (not shown), that correlates each color to each region of the body where the corresponding electrode should be utilized.

As depicted in FIGS. 4, 6 and 7, an optional plurality of adhesives 16 is disposed on the ridged planar surface of the ribbon to create a flat planar surface, similar to that depicted in FIG. 2B. Adhesives 16 may be disposed at set intervals along ribbon 20, or may be disposed in custom configurations as needed or desired by a user. Adhesives 16 may be disposed on only one side of the ridged planar surface of ribbon 20 or on both sides of the ridged planar surface of ribbon 20. A peel away strip (not shown) is disposed in overlying relation to each adhesive 16, so when a user removes the peel away strip (not shown), the adhesive properties of corresponding adhesive 16 are exposed.

Once adhesive properties are exposed, adhesives 16 are capable of securing ribbon 20 to a patient's body and other apparatus in the surgical room, such as the bed frame or other available objects. Thus, adhesives 16 should be of a type that is safe and secure for attachment to both a patient's skin and inanimate apparatus. Securing ribbon 20 to a patient's skin or to surrounding apparatus alleviates the hindrance of ribbon 10 for persons moving around the surgical room. Moreover, if contact is accidentally made with ribbon 20, adhesives 16 alleviate the effect on needles 14, further reducing any artificial waves that may be produced. Contact with wires 2 would pull on the nearest distal adhesive 16 rather than pull on needles 14, allowing needles 14 to remain secure within the patient's body.

Ribbon strand 20 includes electrical connectors 24 at its proximal end. Electrical connectors 24 can be any suitable connection that would connect electrical leads within wire portions 12 to the neurological monitoring apparatus (not shown) that displays electrical activities or evoked potentials/responses transmitted through the electrical leads from the patient's body. Similar to wire portions 12 contiguous with needles 14, wire portions 12 contiguous with electrical connectors 24 are separate and singular, thereby facilitating insertion of electrical connectors 24 into various outlets within the neurological monitoring apparatus. Electrical connectors 24 may include ridges to facilitate gripping and manipulation by the user.

### Example 2

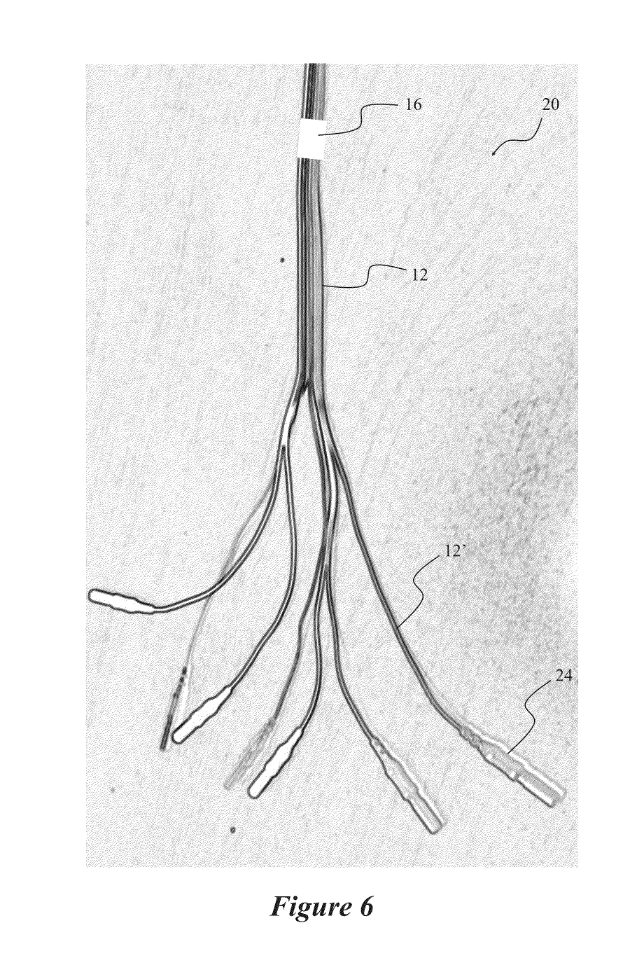

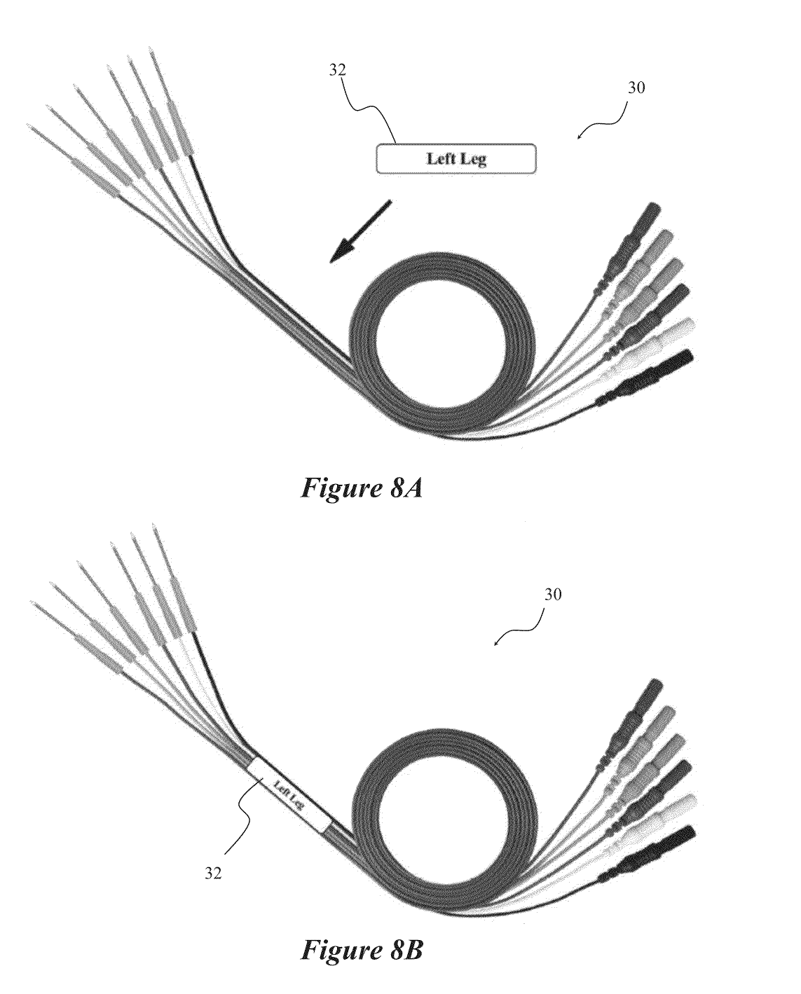

FIGS. 8A-8B depict an alternate embodiment of the current invention where the ribbon strand is denoted generally by the reference numeral 30. As depicted, in addition to or in substitution for the adhesives, one or more labels 32 can be disposed on the ridged planar surface of ribbon 30 to create a flat planar surface, similar to that depicted in FIGS. 4 and 6. Label 32 may be disposed one near either end of ribbon 30, at set intervals along ribbon 30, or in custom configurations as needed or desired by a user. Labels 32 may be disposed on only one side of the ridged planar surface of ribbon 30 or on both sides of the ridged planar surface of ribbon 30.

Labels 32 may be disposed for a variety of purposes, including, but not limited to, blank labels for technician notes before, during or after surgery; labels indicating use, for example “Left Leg” or “Head” as seen in FIGS. 8A and 8B; labels indicating case, for example “Spinal Fusion”' labels indicator the technician or patient, for example “J. Doe” or “1200036”; labels indicating colors, for example “Red=Gastro”; labels indicating intraoperative monitoring group performing or assisting in the surgery; labels indicating user name, for example “ABC Hospital System”; and labels indicating supplier or distributor information, for example “Rochester REF #S86015-R1” or “Reorder from Acme, Inc.”.

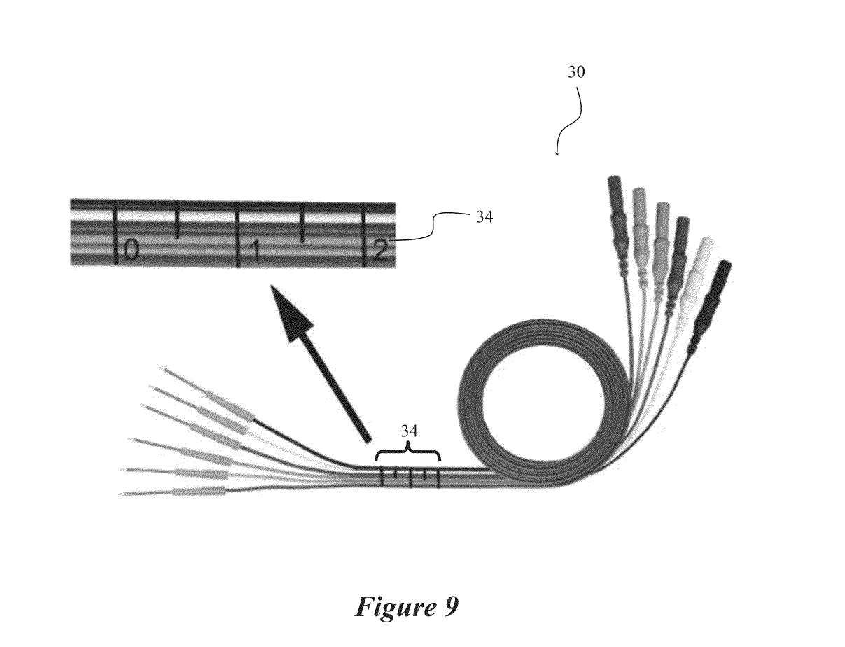

As an alternative option, labels 32 may include ruler 34 (or other measurement markings), or ruler 32 can be printed directly onto the flat planar surface of ribbon strand 30, as seen in FIG. 9.

### DEFINITIONS OF CLAIM TERMS

Customizable: This term is used herein to refer to the ability of an apparatus to be altered or suited to the requirements or specifications of an individual or purpose. For example, a ribbon strand of four or more needle electrodes can be customized for use on a particular individual (i.e., considering height, weight, muscle mass, etc.) undergoing a spinal fusion.

Color-coded: This term is used herein to refer to the ability of an apparatus or portion thereof to indicate information through the use of the color of that apparatus or portion thereof. For example, a ribbon strand can include an array of needle electrodes, and a blue electrode in that array can indicate the position on the patient's body where that electrode should be inserted.

Predetermined dimensions: This term is used herein to refer to a measure of spatial extent of an apparatus in a particular direction, as selected by a user or customized for a surgical procedure. Examples include height, width, length, breadth, depth, etc. For example, a technician can select the length of each needle in a ribbon strand of needle electrodes depending on where each needle needs to be placed in the patient's body for that surgical procedure.

Abuttingly coupled: This term is used herein to refer to the attachment of two apparatuses at a border of each of these apparatuses, such that they are secured adjacent to each other. Two needle electrodes that are abuttingly coupled to each other are attached along the circumferential border of each of the needle electrodes. This can be accomplished via glue or other attachment means.

Diametric opposition: This term is used herein to refer to the relation of opposition along a diameter of a circular apparatus. If two circular apparatuses are abuttingly coupled to each other, another circular apparatus can be abuttingly coupled on the direct opposite side of one of the apparatuses along its diameter (i.e., in diametric opposition) to form a linear formation among all three circular apparatuses.

Manipulation: This term is used herein to refer to an individual's physical control over an apparatus in space.

Proximal: This term is used herein to refer to a spatial position of anything closer to the technician and further from the patient. Thus, the proximal end of a ribbon strand is the end closest to the technician or the computer or other machinery (i.e., the electrical connector).

Distal: This term is used herein to refer to a spatial position of anything closer to the patient and further from the technician. Thus, the distal end of a ribbon strand is the end closest to the patient (i.e., the needle).

Ridged planar surface: This term is used herein to refer to a side of a ribbon strand containing a linear formation of four (4) or more needle electrodes. The ridges are formed by the curvatures of each needle electrode, though the overall formation is linear or flat, providing a plurality of benefits, described previously. Structurally, a ridged planar surface might look similar to that seen in FIG. 2B.

Position of application: This term is used herein to refer to the region of a patient's body where a particular needle electrode is intended to be applied.

It will thus be seen that the objects set forth above, and those made apparent from the foregoing disclosure, are efficiently attained. Since certain changes may be made in the above construction without departing from the scope of the invention, it is intended that all matters contained in the foregoing disclosure or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A customizable ribbon strand of electrodes for use during a surgical procedure, comprising:
a first color-coded needle electrode, said first needle electrode having a first needle with predetermined dimensions, a first wire portion with a first set of predetermined dimensions and having a first electrical lead, and a first electrical connector, wherein said first needle, first wire portion, and first electrical connector maintain electrical flow;
a second color-coded needle electrode, said second needle electrode having a second needle with predetermined dimensions, a second wire portion with a second set of predetermined dimensions and having a second electrical lead, and a second electrical connector, wherein said second needle, second wire portion, and second electrical connector maintain electrical flow;
a third color-coded needle electrode, said third needle electrode having a third needle with predetermined dimensions, a third wire portion with a third set of predetermined dimensions and having a third electrical lead, and a third electrical connector, wherein said third needle, third wire portion, and third electrical connector maintain electrical flow; and
a fourth color-coded needle electrode, said fourth needle electrode having a fourth needle with predetermined dimensions, a fourth wire portion with a fourth set of predetermined dimensions and having a fourth electrical lead, and a fourth electrical connector, wherein said fourth needle, fourth wire portion, and fourth electrical connector maintain electrical flow,
said first needle electrode abuttingly coupled to said second needle electrode, said second needle electrode abuttingly coupled to said third needle electrode in diametric opposition to the coupling position of said first and second needle electrodes, said third needle electrode abuttingly coupled to said fourth needle electrode in diametric opposition to the coupling position of said second and third needle electrodes,
whereby said first, second, third, and fourth needle electrodes form four needle electrodes in said ribbon strand, said four needle electrodes collectively forming a ridged planar surface on each side of said ribbon strand.

2. A ribbon strand as in claim 1, further comprising a plurality of adhesives disposed on at least one side of said ridged planar surface.

3. A ribbon strand as in claim 1, further comprising one or more labels attached on at least one side of said ridged planar surface.

4. A ribbon strand as in claim 1, further comprising a plurality of measurement markings printed along at least one side of said ridged planar surface.

5. A ribbon strand as in claim 1, wherein one of said four needle electrodes having a corresponding needle and wire portion with a different set of predetermined dimensions than at least one of the remaining needle electrodes.

6. A ribbon strand as in claim 1, further comprising:
each of said first, second, third, and fourth needle electrode having a color based on position of application of said each needle electrode;
said first, second, third, and fourth sets of predetermined dimensions of said first, second, third, and fourth needle electrodes, respectively, based on said surgical procedure.

7. A ribbon strand as in claim 6, further comprising said first, second, third, and fourth electrical connectors having respective colors that are respectively the same as said first, second, third, and fourth wire portions, said wire portions being contiguous with said electrical connectors.

8. A ribbon strand as in claim 1, further comprising a distal enclosure disposed in outer relation to a proximal end of each of said first, second, third, and fourth needles and a distal end of each of said first, second, third, and fourth electrical leads within their respective wire portions, whereby said distal enclosure helps secure said needles to said wire portions.

9. A ribbon strand as in claim 8, said distal enclosure having a color that is the same as its respective first, second, third, or fourth wire portion, said respective wire portion being contiguous with said distal enclosure.

10. A ribbon strand as in claim 1, further comprising a needle protector disposed in outer relation to each of said first, second, third, and fourth needles, said needle protector having a length longer than a length of said each needle.

11. A ribbon strand as in claim 1, further comprising said first, second, third, and fourth electrical connectors each having a ridged surface to facilitate manipulation of said each electrical connector by a user.

12. A ribbon strand as in claim 1, further comprising each wire portion of said four needle electrodes separate from said ribbon strand at a proximal end of said ribbon strand and at a distal end of said ribbon strand.

13. A ribbon strand as in claim 1, further comprising a plurality of components disposed on both sides of said ridged planar surface of said ribbon strand to enhance effectiveness of said ribbon strand, said plurality of components selected from the group consisting of adhesives, labels, and printed measurement markings.

14. A ribbon strand as in claim 1, further comprising said needle soldered or crimped to said wire portion.

15. A ribbon strand as in claim 1, further comprising:
a fifth color-coded needle electrode, said fifth needle electrode having a fifth needle with predetermined dimensions, a fifth wire portion with a fifth set of predetermined dimensions and having a fifth electrical lead, and a fifth electrical connector, wherein said fifth needle, fifth wire portion, and fifth electrical connector maintain electrical flow,
said fifth needle electrode abuttingly coupled to said fourth needle electrode in diametric opposition to the coupling position of said third and fourth needle electrodes.

16. A method of manufacturing a ribbon strand of electrodes for use during a surgical procedure, comprising the steps of:
identifying said surgical procedure;
determining a number of needle electrodes needed for said surgical procedure, said number being four or more needle electrodes, said needle electrodes each including a needle and a wire portion with electrical lead;
determining a needle length of said each needle electrode based on said surgical procedure;
determining a wire portion length of said each needle electrode based on said surgical procedure;
coupling a first needle electrode in abutting relation to a second needle electrode;
coupling said second needle electrode in abutting relation to a third needle electrode in diametric opposition to the coupling of said first needle electrode to said second needle electrode; and
coupling said third needle electrode in abutting relation to a fourth needle electrode in diametric opposition to the coupling of said second needle electrode to said third needle electrode, such that said four or more needle electrodes form a ridged planar surface on each side of said ribbon strand.

17. A method of manufacturing a ribbon strand of electrodes as in claim 16, further comprising the step of attaching a plurality of adhesives on at least one side of said ridged planar surface.

18. A method of manufacturing a ribbon strand of electrodes as in claim 16, further comprising the step of attaching one or more labels on at least one side of said ridged planar surface.

19. A method of manufacturing a ribbon strand of electrodes as in claim 16, further comprising the step of printing measurement markings directly onto at least one side of said ridged planar surface.

20. A method of manufacturing a ribbon strand of electrodes as in claim 16, further comprising the step of soldering or crimping said needle to said wire portion of said each needle electrode.

21. A method of manufacturing a ribbon strand of electrodes as in claim 16, further comprising the step of disposing a color on said each needle electrode based on position of application of said each needle electrode in said identified surgical procedure.
